(19) United States
(12) Patent Application Publication
(10) Pub. No.: US 2006/0189835 A1
(43) Pub. Date: Aug. 24, 2006

(54) Method for producing 1,1,1-trifluoroethane

(75) Inventor: Veronique Mathieu, Wavre (FR)

Correspondence Address: CONNOLLY BOVE LODGE & HUTZ, LLP, P O BOX 2207, WILMINGTON, DE, 19899, US

(73) Assignee: SOLVAY S.A., Brussels (BE)

(21) Appl. No.: 10/548,338

(22) PCT Filed: Mar. 5, 2004
(86) PCT No.: PCT/EP04/02499

§ 371 Date: Feb. 10, 2006

(30) Foreign Application Priority Data
Mar. 7, 2003 (FR) 03/03027

(51) Int. Cl. C07C 17/08

(52) U.S. Cl. 570/169

(57) ABSTRACT

Process for the manufacture of 1,1,1-trifluoroethane (HFC-143a), according to which 1,1-difluoro-1-chloroethane (HCFC-142b) is subjected to a vapour-phase reaction with hydrogen fluoride (HF) in the presence of a hydrofluorination catalyst, in which process the molar ratio of the HCFC-142b introduced to the HF introduced into the vapour-phase reaction is greater than or equal to 1 and less than 1.3.

The present invention relates to a process for the manufacture of 1,1,1-trifluoroethane (HFC-143a).

HFC-143a is used as constituent of refrigerant mixtures which are replacing chlorofluorocarbons.

Patent Application EP-A-714 874 discloses the manufacture of HFC-143a from 1,1-difluoro-1-chloroethane (HCFC-142b) in the vapour phase with an HF/HCFC-142b molar ratio of greater than 1. European Patent EP-B-714 874 teaches that this ratio has to be at least 1.3 in order to avoid the formation of by-products by decomposition of the HCFC-142b.

It was desirable to make available a selective process for the manufacture of HFC-143a from HCFC-142b which makes it possible to achieve a good productive output by volume and which minimizes the need for purification operations on conclusion of the hydrofluorination reaction.

The invention consequently relates to a process for the manufacture of 1,1,1-trifluoroethane (HFC-143a), according to which 1,1-difluoro-1-chloro-ethane (HCFC-142b) is subjected to a vapour-phase reaction with hydrogen fluoride (HF) in the presence of a hydrofluorination catalyst, in which process the molar ratio of the HCFC-142b introduced to the HF introduced into the vapour-phase reaction is greater than or equal to 1 and less than 1.3.

It has been found, surprisingly and contrary to the teaching of the document EP 714 874, that it is possible to efficiently and selectively obtain HFC-143a from HCFC-142b while operating with an HF/HCFC-142b ratio of approximately 1. In the process according to the invention, good stability with regard to the activity of the catalyst over time is also observed.

In the process according to the invention, the HF/HCFC-142b molar ratio is often greater than or equal to 1.02. Preferably, this molar ratio is greater than or equal to 1.05. In the process according to the invention, the HF/HCFC-142b molar ratio is often less than or equal to 1.25. Preferably, this molar ratio is less than or equal to 1.20.

In the process according to the invention, the temperature is generally greater than or equal to 100° C. Preferably, the temperature is greater than or equal to 150° C. In the process according to the invention, the temperature is generally less than or equal to 400° C. Preferably, the temperature is less than or equal to 250° C.

In the process according to the invention, the pressure is generally greater than or equal to 1 bar. Preferably, the pressure is greater than or equal to 5 bar. In the process according to the invention, the pressure is generally less than or equal to 30 bar. Preferably, the pressure is less than or equal to 15 bar.

In the process according to the invention, the contact time, defined as the ratio of the volume of the catalyst to the flow rate of HF and HCFC-142b introduced into the reactor, is generally greater than or equal to 1 s. Preferably, the contact time is greater than or equal to 10 s. In the process according to the invention, the contact time, defined as the ratio of the volume of the catalyst to the flow rate of HF and HCFC-142b introduced into the reactor, is generally less than or equal to 200 s. Preferably, the contact time is less than or equal to 50 s.

In the process according to the invention, the hydrofluorination catalyst can be chosen, for example, from supported or unsupported metal salts. If appropriate, the support can, for example, be active charcoal.

The hydrofluorination catalyst can advantageously comprise chromium oxide. An amorphous chromium oxide exhibiting, before an optional prefluorination treatment, a BET/N₂ specific surface of greater than or equal to 100 m²/g gives good results. An amorphous chromium oxide exhibiting such a specific surface of greater than or equal to 200 m²/g is preferred. If appropriate, the amorphous chromium oxide generally exhibits, before an optional prefluorination treatment, a BET/N₂ specific surface of less than or equal to 600 m²/g, preferably of less than or equal to 400 m²/g.

A catalyst which is particularly preferred for use in the process according to the invention comprises chromium and magnesium. This catalyst can be obtained by a method according to which:

(a) a water-soluble chromium(III) salt is reacted with magnesium hydroxide or magnesium oxide, and optionally graphite, in the presence of water;

(b) the reaction mixture obtained is converted into a paste;

(c) the paste is dried;

(d) the dried paste is subjected to treatment with hydrogen fluoride at temperatures of 20 to 500° C;

and the amounts of water-soluble chromium(III) salt, of magnesium hydroxide or of magnesium oxide, and optionally of graphite, are respectively chosen so that the dried paste obtained in stage (c) comprises from 3.5 to 26% by weight, preferably from 4.5 to 23% by weight, of chromium, expressed in the form of Cr₂O₃, at least 25% by weight of magnesium, expressed in the form of MgO, and optionally graphite, preferably in an amount of 5 to 40% by weight. The manufacture of such a catalyst is disclosed, for example, in Application EP-A-733 611, the content of which with regard to this subject is incorporated by reference in the present patent application.

In another aspect, it has been found that, with the catalyst comprising chromium and magnesium described above, all other characteristics remaining as defined above, specific advantages for stability of the activity of the catalyst are also obtained when the molar ratio of the HCFC-142b introduced to the HF introduced into the vapour-phase reaction is greater than or equal to 1.3. In this specific aspect, such a ratio of greater than or equal to 2 can be employed. In this specific aspect, the molar ratio of the HCFC-142b introduced to the HF introduced into the vapour-phase reaction is generally less than or equal to 10. Preferably, in this specific aspect, this ratio is less than or equal to 5.

The HCFC-142b used as starting material in the process according to the invention is available commercially. Alternatively, it can be obtained by hydrofluorination starting from vinylidene chloride or from 1,1,1-trichloroethane or their mixtures.

The stream of reactants introduced into the vapour-phase reaction is preferably composed essentially of HCFC-142b and of hydrogen fluoride.

Other compounds, such as in particular 1,1-dichloro-1-fluoroethane, can optionally be present in the stream of reactants introduced into the vapour-phase reaction. Preferably, the content of such compounds is less than 5 mol %, with respect to the sum of moles of compounds present in the stream of reactants. A content of less than 1 mol% is more particularly preferred.

The process according to the invention can be carried out continuously or batchwise. A continuous process is preferred.

The process according to the invention can be carried out in any reactor appropriate for carrying out a vapour-phase hydrofluorination process. Mention may in particular be made of a tubular reactor, made of materials resistant to the presence of HF at the temperature and pressure of the reaction, comprising a fixed bed of catalyst.

The example below is intended to illustrate the invention without, however, limiting it.

### EXAMPLE

A catalyst comprising 4.6% by weight of Cr, expressed in the form of Cr₂O₃, Mg and graphite, obtained in accordance with the example of the document EP 733 611, was introduced into a tubular reactor with a volume of 70 ml made of Hastelloy B2.

The catalyst was dried at 150° C. for 1 hour under a flushing stream of nitrogen at a flow rate of 10 N₂/h. The catalyst was subsequently fluorinated with an HF/N₂ mixture (11 l HF/h-20 l N₂/h) at 200° C. for 1 hour and then at 250° C. for 1 hour, at 300° C. for 6 hours and at 350° C. for 8 hours.

After fluorination, heating was carried out to a temperature of 200° C. HCFC-142b and HF were introduced continuously. The HF/HCFC-142b ratio introduced was 1.1. The pressure of the reaction was 10 bar. On conclusion of the reaction, a gas phase comprising HFC-143a was recovered and was introduced into a washing column in order to remove, by washing with an aqueous KOH solution, the excess HF and the HCl produced. The gas exiting from this washing operation was analysed by gas chromatography. The conversion of HCFC-142b was 93.5% and the selectivity for HFC-143a was 99.5%. The 0.5% of impurities comprised 50% of HCFC-141b. The reaction was continued for 600 h without loss of activity or of selectivity.

1. Process for the manufacture of 1,1,1-trifluoroethane (HFC-143a), according to which 1,1-difluoro-1-chloroethane (HCFC-142b) is subjected to a vapour-phase reaction with hydrogen fluoride (HF) in the presence of a hydrofluorination catalyst, in which process the molar ratio of the HCFC-142b introduced to the HF introduced into the vapour-phase reaction is greater than or equal to 1 and less than 1.3.

2. Process according to claim 1, in which the molar ratio is greater than or equal to 1.02.

3. Process according to claim 2, in which the molar ratio is greater than or equal to 1.05.

4. Process according to claim 1, in which the molar ratio is greater than or equal to 1.25.

5. Process according to claim 4, in which the molar ratio is greater than or equal to 1.20.

6. Process according to claim 1, in which the temperature of the reaction is from 100 to 400° C.

7. Process according to claim 1, in which the pressure of the reaction is from 1 to 30 bar.

8. Process according to claim 1, in which the contact time is from 1 to 200 s.

9. Process according to claim 1, in which the hydrofluorination catalyst comprises chromium oxide.

10. Process according to claim 9, in which the catalyst comprises chromium and magnesium and the catalyst can be obtained by a method according to which:
(a) a water-soluble chromium(III) salt is reacted with magnesium hydroxide or magnesium oxide, and optionally graphite, in the presence of water;
(b) the reaction mixture obtained is converted into a paste;
(c) the paste is dried;
(d) the dried paste is subjected to treatment with hydrogen fluoride at temperatures of 20 to 500° C.;
and the amounts of water-soluble chromium(III) salt and of magnesium hydroxide or of magnesium oxide are respectively chosen so that the dried paste obtained in stage (c) comprises from 3.5 to 26% by weight of chromium, expressed in the form of Cr₂O₃, and at least 25% by weight of magnesium, expressed in the form of MgO.

11. Process according to claim 3, in which the molar ratio is greater than or equal to 1.25.

12. Process according to claim 11, in which the molar ratio is greater than or equal to 1.20.

13. Process according to claim 12, in which the temperature of the reaction is from 100 to 400° C.

14. Process according to claim 13, in which the pressure of the reaction is from 1 to 30 bar.

15. Process according to claim 14, in which the contact time is from 1 to 200 s.

16. Process according to claim 15, in which the hydrofluorination catalyst comprises chromium oxide.
